(19) United States
(12) Patent Application Publication
(10) Pub. No.: US 2026/0069118 A1
(43) Pub. Date: Mar. 12, 2026

(54) ENDOSCOPE-GUIDED PERCUTANEOUS VISIBLE DEVICE FOR ABDOMINAL CAVITY PROBING AND ACCURATE DRAINAGE

(71) Applicant: Zhaojun XU, Ningbo (CN)

(72) Inventor: Zhaojun XU, Ningbo (CN)

(21) Appl. No.: 18/963,228

(22) Filed: Nov. 27, 2024

(30) Foreign Application Priority Data
Sep. 6, 2024 (CN) 202411245857X

(51) Int. Cl. A61B 1/015; A61B 1/00

(52) U.S. Cl. CPC A61B 1/015; A61B 1/00045

(57) ABSTRACT

The present invention discloses an endoscope-guided percutaneous visible device for abdominal cavity probing and accurate drainage and relates to the technical field of abdominal cavity drainage devices. The device includes a drainage tube, where an end surface of a bottom end of the drainage tube is provided with a plurality of through holes, and a fluid injection tube and a fluid discharge tube are disposed on a circumferential surface of a top end of the drainage tube in a communicated manner. The device further includes: a data line, fixedly disposed in the drainage tube; and a camera, fixedly mounted on an end surface of a bottom end of the data line, where a display mechanism is disposed at a top end of the data line, and the display mechanism includes a handgrip.

### CROSS-REFERENCE TO RELATED APPLICATIONS

The application claims priority to Chinese patent application No. 202411245857X, filed on Sep. 6, 2024, the entire contents of which are incorporated herein by reference.

### TECHNICAL FIELD

The present invention relates to the technical field of abdominal cavity drainage devices, and specifically, to an endoscope-guided percutaneous visible device for abdominal cavity probing and accurate drainage.

### BACKGROUND

Abdominal cavity surgery refers to the process of entering an abdominal cavity through surgery to diagnose, treat, or repair organs or tissue in the abdominal cavity. This surgery can be performed through traditional open surgery or more minimally invasive laparoscopic surgery. Abdominal cavity drainage surgery refers to draining effusions, pus, blood, and other fluids in the abdominal cavity out of the body by placing a drainage tube, thereby reducing pressure and fluid accumulation in the abdominal cavity and preventing complications.

However, at present, a bedside abdominal cavity drainage tube device is usually placed blindly, so that an actual position of a catheter head cannot be seen, and a local lesion cannot be accurately located. Therefore, currently, most surgical operations are performed through exploratory celiotomy, resulting in large surgical trauma. In addition, the current abdominal cavity drainage tube device cannot achieve visible flushing and drainage, resulting in drainage deviation easily.

### SUMMARY

A purpose of the present invention is to provide an endoscope-guided percutaneous visible device for abdominal cavity probing and accurate drainage, to solve the above deficiencies in the prior art.

To implement the above purpose, the present invention provides the following technical solutions: an endoscope-guided percutaneous visible device for abdominal cavity probing and accurate drainage, including a drainage tube, where an end surface of a bottom end of the drainage tube is provided with a plurality of through holes, and a fluid injection tube and a fluid discharge tube are disposed on a circumferential surface of a top end of the drainage tube in a communicated manner; and further including: a data line, fixedly disposed in the drainage tube; and a camera, fixedly mounted on an end surface of a bottom end of the data line, where a display mechanism is disposed at a top end of the data line; and the display mechanism includes a handgrip, electrically connected to an end surface of the top end of the data line, and further includes a display screen, rotatably disposed at a top end of the handgrip through an adjusting mechanism.

Further, the top end of the drainage tube is provided with a first fixing port in a communicated manner, and the data line is disposed in the first fixing port.

Further, a connecting head is disposed on an end surface of one end, away from the drainage tube, of the fluid injection tube in a communicated manner, a connecting tube is disposed on the connecting head in a communicated manner, and an injection syringe is disposed on the connecting tube in a communicated manner.

Further, a sealing ring is disposed between the connecting head and the connecting tube, and the sealing ring is made of rubber.

Further, an end surface of one end, away from the drainage tube, of the fluid discharge tube is provided with a second fixing port in a communicated manner, and a waste fluid bag is disposed on the second fixing port.

Further, a protective head is disposed on the end surface of the bottom end of the drainage tube in a threaded manner, and the protective head is made of rubber.

Further, a protective cover is disposed in the protective head, the protective cover is disposed on the end surface of the bottom end of the data line in a clamping manner, and the camera is disposed in the protective cover.

Further, the adjusting mechanism includes a fixing base fixedly mounted at the top end of the handgrip, a support plate is fixedly mounted on a top surface of the fixing base, a worm is rotatably disposed on the support plate, and a first bevel gear is fixedly mounted at one end of the worm. A first rotating rod is rotatably disposed on the fixing base, a second bevel gear engaged with the first bevel gear is fixedly mounted at a top end of the first rotating rod, a first circular gear is fixedly mounted at a bottom end of the first rotating rod, and a first gear plate engaged with the first circular gear is rotatably disposed on a circumferential surface of the handgrip. A rotating frame is further rotatably disposed on the support plate, a rotating shaft is rotatably disposed on the rotating frame, and a worm wheel matched with the worm is fixedly mounted on a circumferential surface of the rotating shaft. A mounting rack is rotatably mounted on end surfaces of two ends of the rotating shaft, and the mounting rack is fixedly mounted at a bottom end of the display screen.

Further, a second rotating rod is further rotatably disposed on the fixing base, a third bevel gear is fixedly mounted at a top end of the second rotating rod, and a fourth bevel gear engaged with the third bevel gear is fixedly mounted on a side wall of the rotating frame. A second circular gear is fixedly mounted at a bottom end of the second rotating rod, and a second gear plate engaged with the second circular gear is further rotatably disposed on a circumferential surface of the handgrip.

Further, a first knob and a second knob are further rotatably disposed on the circumferential surface of the handgrip, the first knob and the second knob are fixedly connected to the first gear plate and the second gear plate respectively, and the second knob is rotatably sleeved on the first knob.

In the above technical solutions, the present invention provides an endoscope-guided percutaneous visible device for abdominal cavity probing and accurate drainage and has the following beneficial effects:

1. By using the camera in the drainage tube, the drainage tube can enter an abdominal cavity of a patient by passing through a wound, and by using the display mechanism, information probed by using the camera is fed back to the display screen through the data line, thereby facilitating probing the abdominal cavity of the patient by a medical worker, so that a surgical wound is reduced, and safety during surgery is improved.

2. By using the fluid injection tube and the drainage tube, liquid medicine can flow into a body of the patient through the fluid injection tube and the drainage tube, thereby facilitating flushing the interior of the wound of the patient by the medical worker. By using the fluid discharge tube and the drainage tube, the medical worker can discharge the liquid medicine after flushing the patient.

3. By using the adjusting mechanism, the display screen on the top end of the handgrip can be adjusted, thereby avoiding, as much as possible, a situation that the display screen is blurred due to illumination of light on the display screen during the surgery, causing unclear viewing for the medical worker.

4. Therefore, the camera is used to probe the interior of an entire abdominal cavity, so that placement of the drainage tube of the abdominal cavity is visible, a local lesion can be accurately located, exploratory celiotomy can be replaced, various difficult causes can be identified, to significantly reduce surgical trauma. In addition, visible flushing and drainage are performed, so that drainage becomes very accurate.

### BRIEF DESCRIPTION OF THE DRAWINGS

To describe the technical solutions in the embodiments of the present application or in the prior art more clearly, the following briefly introduces the accompanying drawings required for describing the embodiments. Apparently, the accompanying drawings in the following description show merely some embodiments of the present invention, and those of ordinary skill in the art may still derive other drawings from these accompanying drawings.

FIG. 1 is a schematic diagram of an overall structure according to an embodiment of the present invention;

FIG. 2 is a schematic diagram of a partial structure of FIG. 1 according to an embodiment of the present invention;

FIG. 3 is a schematic structural diagram of a bottom end of a drainage tube in FIG. 1 according to an embodiment of the present invention;

FIG. 4 is a schematic exploded view of a partial structure of FIG. 3 according to an embodiment of the present invention;

FIG. 5 is a schematic structural diagram of a position A in FIG. 4 according to an embodiment of the present invention;

FIG. 6 is a schematic structural diagram of a display mechanism in FIG. 1 according to an embodiment of the present invention;

FIG. 7 is a schematic diagram of a partial structure of FIG. 6 according to an embodiment of the present invention;

FIG. 8 is a schematic structural front view of FIG. 6 according to an embodiment of the present invention;

FIG. 9 is a schematic diagram of a partial structure of FIG. 8 according to an embodiment of the present invention; and FIG. 10 is a schematic structural diagram of a first knob and a second knob in FIG. 7 according to an embodiment of the present invention.

### REFERENCE NUMERALS IN THE DRAWINGS

11. drainage tube; 12. first fixing port; 13. through hole; 14. data line; 15. camera; 16. protective head; 17. protective cover; 21. fluid injection tube; 22. connecting head; 23. connecting tube; 24. injection syringe; 31. fluid discharge tube; 32. second fixing port; 4. display mechanism; 41. handgrip; 42. display screen; 5. adjusting mechanism; 501. fixing base; 502. support plate; 503. worm; 504. first bevel gear; 505. first rotating rod; 506. second bevel gear; 507. first circular gear; 508. first gear plate; 509. rotating frame; 510. rotating shaft; 511. worm wheel; 512. mounting rack; 513. second rotating rod; 514. third bevel gear; 515. fourth bevel gear; 516. second circular gear; 517. second gear plate; 518. first knob; 519. second knob.

### DETAILED DESCRIPTION OF THE EMBODIMENTS

To enable a person skilled in the art to better understand the technical solutions in the embodiment of the present invention, the following further describes the present invention in detail with reference to the accompanying drawings.

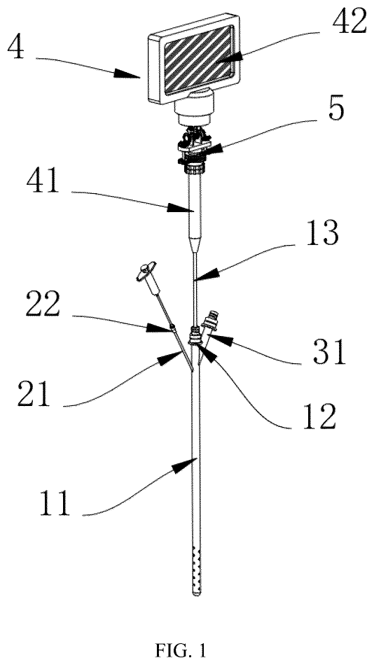

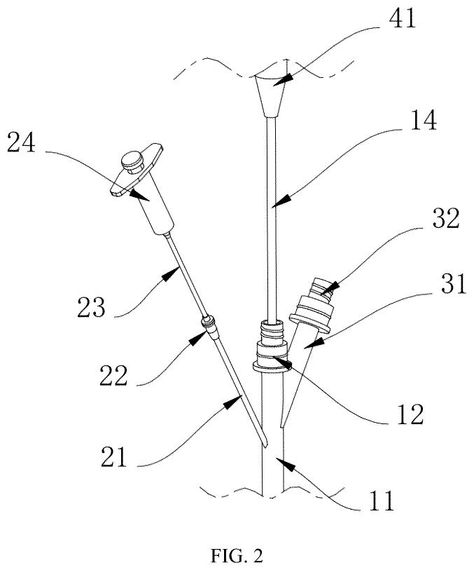

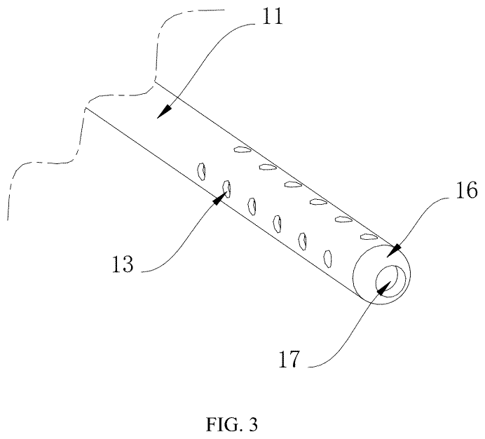

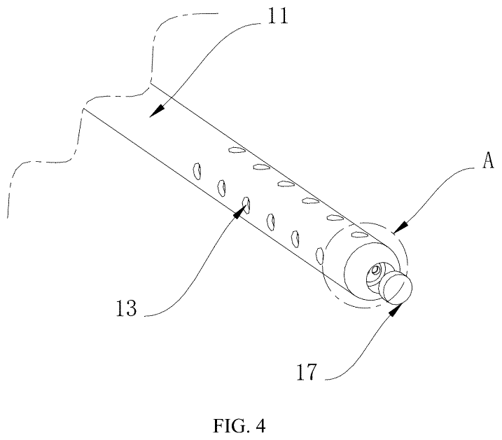

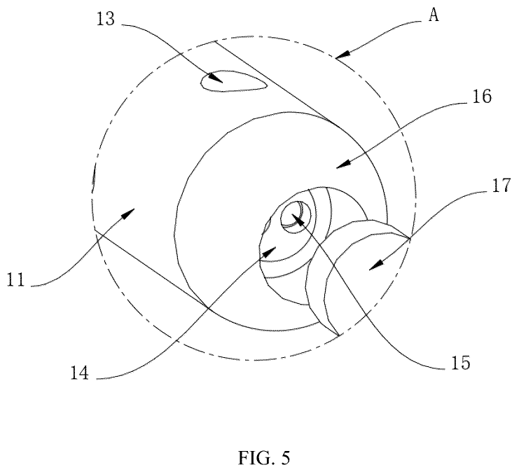

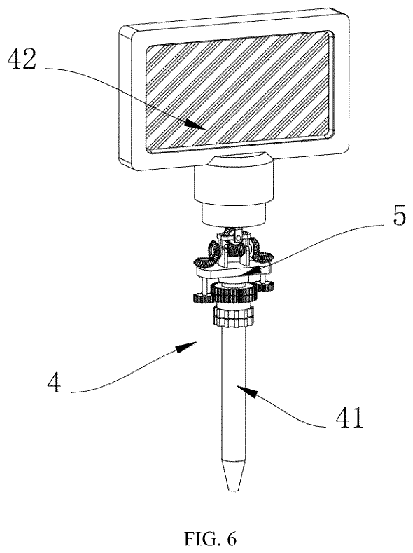

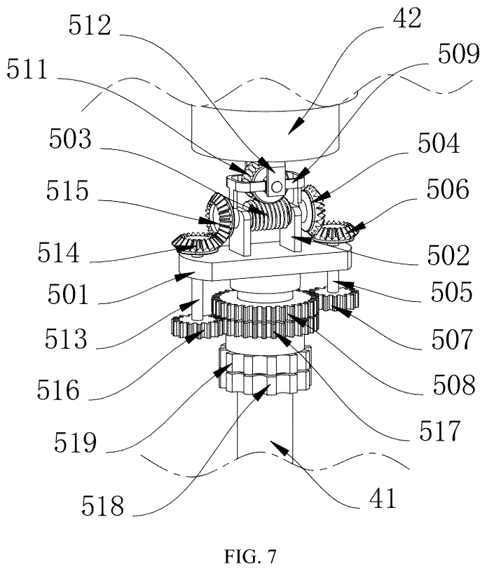

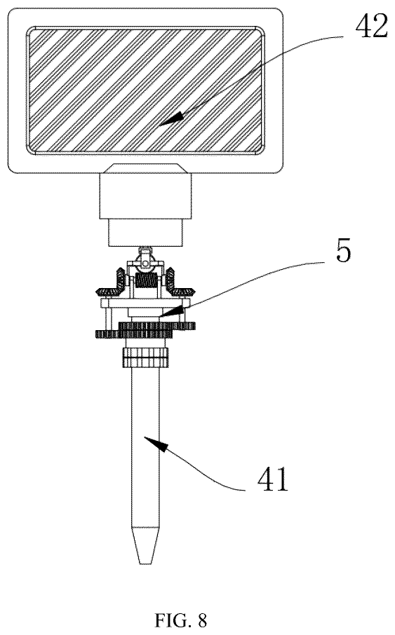

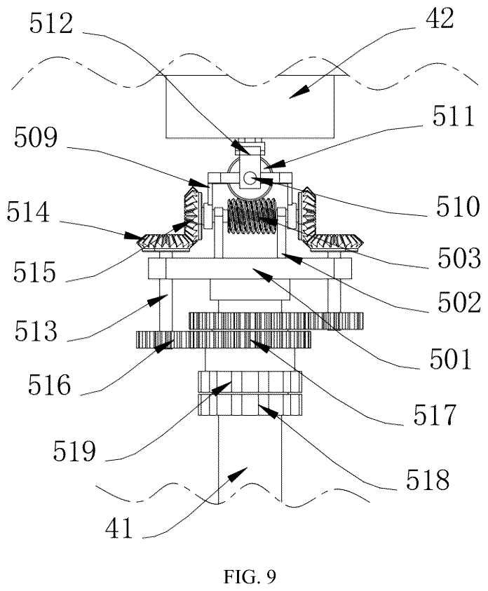

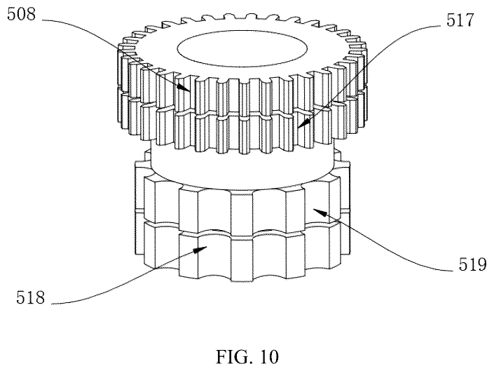

Referring to FIG. 1 to FIG. 10, an endoscope-guided percutaneous visible device for abdominal cavity probing and accurate drainage provided in the embodiment of the present invention includes a drainage tube 11, where an end surface of a bottom end of the drainage tube 11 is provided with a plurality of through holes 13, and a fluid injection tube 21 and a fluid discharge tube 31 are disposed on a circumferential surface of a top end of the drainage tube 11 in a communicated manner. In the above technical solution, by using the fluid injection tube 21 and the drainage tube 11, liquid medicine can flow into a body of a patient through the fluid injection tube 21 and the drainage tube 11, thereby facilitating flushing the interior of a wound of the patient by a medical worker. By using the fluid discharge tube 31 and the drainage tube 11, the medical worker can discharge the liquid medicine after flushing the patient. The device further includes: a data line 14, fixedly disposed in the drainage tube 11; and a camera 15, fixedly mounted on an end surface of a bottom end of the data line 14, where a display mechanism 4 is disposed at a top end of the data line 14; and the display mechanism 4 includes a handgrip 41, electrically connected to an end surface of the top end of the data line 14, and further includes a display screen 42, rotatably disposed at a top end of the handgrip 41 through an adjusting mechanism 5. In the above technical solution, by using the camera 15 in the drainage tube 11, the drainage tube 11 can enter an abdominal cavity of the patient by passing through the wound, and by using the display mechanism 4, information probed by using the camera 15 is fed back to the display screen 42 through the data line 14, thereby facilitating probing the abdominal cavity of the patient by the medical worker, so that a surgical wound is reduced, and safety during surgery is improved. By using the adjusting mechanism 5, the display screen 42 at the top end of the handgrip 41 can be adjusted, thereby avoiding, as much as possible, a situation that the display screen 42 is blurred due to illumination of light on the display screen 42 during the surgery, causing unclear viewing for the medical worker.

Specifically, the top end of the drainage tube 11 is provided with a first fixing port 12 in a communicated manner, and the data line 14 is disposed in the first fixing port 12.

In the above technical solution, by using the first fixing port 12, the data line 14 can be fixed, thereby avoiding, as much as possible, a situation that normal use of the drainage tube 11 is affected due to looseness of the data line 14 during use of the drainage tube 11.

Specifically, a connecting head 22 is disposed on an end surface of one end, away from the drainage tube 11, of the fluid injection tube 21 in a communicated manner, a connecting tube 23 is disposed on the connecting head 22 in a communicated manner, and an injection syringe 24 is disposed on the connecting tube 23 in a communicated manner.

In the above technical solution, by using the injection syringe 24, the liquid medicine can flow into the body of the patient along the connecting tube 23, a fluid inlet tube, and the drainage tube 11 by means of pressing of the injection syringe 24, thereby facilitating flushing the patient by the medical worker.

Specifically, a sealing ring is disposed between the connecting head 22 and the connecting tube 23, and the sealing ring is made of rubber.

In the above technical solution, by using the sealing ring, sealing between the connecting tube 23 and a connecting portion of the fluid inlet tube can be improved, thereby avoiding, as much as possible, a situation that the liquid medicine seeps out.

Specifically, an end surface of one end, away from the drainage tube 11, of the fluid discharge tube 31 is provided with a second fixing port 32 in a communicated manner, and a waste fluid bag is disposed on the second fixing port 32.

In the above technical solution, after the wound of the patient is flushed, by using negative pressure of the waste fluid bag, the flushed liquid medicine can flow along the drainage tube 11 and the fluid discharge tube 31 under the negative pressure, to flow into the waste fluid bag, thereby recycling the flushed liquid medicine.

Specifically, a protective head 16 is disposed on the end surface of the bottom end of the drainage tube 11 in a threaded manner, and the protective head 16 is made of rubber.

In the above technical solution, by using the protective head 16 made of a soft material, flexibility of the end surface of the drainage tube 11 can be improved, thereby avoiding, as much as possible, a situation that when the drainage tube 11 passes through a minimally invasive wound, the wound is scratched to cause secondary damage to the wound due to a hard port of the drainage tube 11.

Specifically, a protective cover 17 is disposed in the protective head 16, the protective cover 17 is disposed on the end surface of the bottom end of the data line 14 in a clamping manner, and the camera 15 is disposed in the protective cover 17.

In the above technical solution, by using the protective cover 17, the camera 15 can be protected.

In abdominal cavity surgery, due to illumination of light in an operating room on the display screen 42, the display screen 42 is easily blurred under direct illumination of the light. Since the display screen 42 is blurred, the medical worker cannot clearly and effectively view the interior of the abdominal cavity of the patient through the display screen 42. Therefore, the medical worker needs to adjust an angle of the display screen 42, so that the medical worker can effectively view the display screen 42. However, in the prior art, since the medical worker needs to operate the drainage tube 11 to perform the operation by using one hand, it is not convenient for the other hand holding the handgrip 41 to perform one-hand adjustment on the angle of the display screen 42, and therefore, an abdominal cavity surgery operation is affected.

Specifically, the adjusting mechanism 5 includes a fixing base 501 fixedly mounted at the top end of the handgrip 41, a support plate 502 is fixedly mounted on a top surface of the fixing base 501, a worm 503 is rotatably disposed on the support plate 502, and a first bevel gear 504 is fixedly mounted at one end of the worm 503. A first rotating rod 505 is rotatably disposed on the fixing base 501, a second bevel gear 506 engaged with the first bevel gear 504 is fixedly mounted at a top end of the first rotating rod 505, a first circular gear 507 is fixedly mounted at a bottom end of the first rotating rod 505, and a first gear plate 508 engaged with the first circular gear 507 is rotatably disposed on a circumferential surface of the handgrip 41. A rotating frame 509 is further rotatably disposed on the support plate 502, a rotating shaft 510 is rotatably disposed on the rotating frame 509, and a worm wheel 511 matched with the worm 503 is fixedly mounted on a circumferential surface of the rotating shaft 510. A mounting rack 512 is rotatably mounted on end surfaces of two ends of the rotating shaft 510, and the mounting rack 512 is fixedly mounted at a bottom end of the display screen 42.

Further, a second rotating rod 513 is further rotatably disposed on the fixing base 501, a third bevel gear 514 is fixedly mounted at a top end of the second rotating rod 513, and a fourth bevel gear 515 engaged with the third bevel gear 514 is fixedly mounted on a side wall of the rotating frame 509. A second circular gear 516 is fixedly mounted at a bottom end of the second rotating rod 513, and a second gear plate 517 engaged with the second circular gear 516 is further rotatably disposed on a circumferential surface of the handgrip 41.

In the above technical solution, by using the first gear plate 508 and through mutual matching between the first gear plate and the first circular gear 507 on the first rotating rod 505, the second bevel gear 506 on the first rotating rod 505 is rotated. Through mutual matching between the first bevel gear 504 and the second bevel gear 506, the worm 503 on the support plate 502 is rotated. Through mutual matching between the worm 503 and the worm wheel 511, the display screen 42 can swing leftward and rightward.

By using the second gear plate 517 and through mutual matching between the second gear plate and the second circular gear 516 on the second rotating rod 513, the fourth bevel gear 515 on the second rotating rod 513 is rotated. Through mutual matching between the third bevel gear 514 and the fourth bevel gear 515, the rotating frame 509 on the support plate 502 is rotated, so that the display screen 42 can swing forward and backward.

Therefore, through mutual matching between the above structures, the medical worker may adjust the angle of the display screen 42 on the handgrip 41 with one hand only, thereby improving convenience of the display screen 42.

Specifically, a first knob 518 and a second knob 519 are further rotatably disposed on the circumferential surface of the handgrip 41, the first knob 518 and the second knob 519 are fixedly connected to the first gear plate 508 and the second gear plate 517 respectively, and the second knob 519 is rotatably sleeved on the first knob 518.

In the above technical solution, by using the first knob 518 and the second knob 519, the first gear plate 508 and the second gear plate 517 can be operated conveniently to rotate.

A working principle: after minimally invasive wound opening is performed on the abdominal cavity of the patient, by using the drainage tube 11, the drainage tube 11 enters the abdominal cavity of the patient by passing through the minimally invasive wound, and by using the display mechanism 4, the information probed by using the camera 15 is fed back to the display screen 42 thought the data line 14. Therefore, the medical worker probes the abdominal cavity of the patient.

When the wound of the abdominal cavity of the patient needs to be cleaned, by using the injection syringe 24, the liquid medicine can flow into the body of the patient along the connecting tube 23, the fluid inlet tube, and the drainage tube 11 by means of the pressing of the injection syringe 24, thereby facilitating flushing the interior of the abdominal cavity of the patient by the medical worker. After the wound of the patient is flushed, by using the negative pressure of the waste fluid bag, the flushed liquid medicine can flow along the drainage tube 11 and the fluid discharge tube 31 under the negative pressure, to flow into the waste fluid bag, thereby recycling the flushed liquid medicine.

When the angle of the display screen 42 on the handgrip 41 needs to be adjusted, by using the first gear plate 508 and through the mutual matching between the first gear plate and the first circular gear 507 on the first rotating rod 505, the second bevel gear 506 on the first rotating rod 505 is rotated. Through the mutual matching between the first bevel gear 504 and the second bevel gear 506, the worm 503 on the support plate 502 is rotated. Through the mutual matching between the worm 503 and the worm wheel 511, the display screen 42 can swing leftward and rightward. By using the second gear plate 517 and through the mutual matching between the second gear plate and the second circular gear 516 on the second rotating rod 513, the fourth bevel gear 515 on the second rotating rod 513 is rotated. Through the mutual matching between the third bevel gear 514 and the fourth bevel gear 515, the rotating frame 509 on the support plate 502 is rotated, so that the display screen 42 can swing forward and backward. Therefore, through the mutual matching between the above structures, the medical worker may adjust the angle of the display screen 42 on the handgrip 41 with one hand only, thereby improving convenience of the display screen 42.

The above descriptions only describe some exemplary embodiments of the present invention by way of illustration. It is undoubted that those of ordinary skill in the art can modify the described embodiments in various ways without departing from the spirit and scope of the present invention. Therefore, the above accompanying drawings and descriptions are illustrative in nature and should not be construed as limiting the protection scope of the claims of the present invention.

What is claimed is:

1. An endoscope-guided percutaneous visible device for abdominal cavity probing and accurate drainage, comprising a drainage tube (11), wherein an end surface of a bottom end of the drainage tube (11) is provided with a plurality of through holes (13), and a fluid injection tube (21) and a fluid discharge tube (31) are disposed on a circumferential surface of a top end of the drainage tube (11) in a communicated manner; and further comprising:
a data line (14), fixedly disposed in the drainage tube (11); and
a camera (15), fixedly mounted on an end surface of a bottom end of the data line (14), wherein a display mechanism (4) is disposed at a top end of the data line (14); and
the display mechanism (4) comprises a handgrip (41), electrically connected to an end surface of the top end of the data line (14), and further comprises a display screen (42), rotatably disposed at a top end of the handgrip (41) through an adjusting mechanism (5).

2. The endoscope-guided percutaneous visible device for abdominal cavity probing and accurate drainage according to claim 1, wherein the top end of the drainage tube (11) is provided with a first fixing port (12) in a communicated manner, and the data line (14) is disposed in the first fixing port (12).

3. The endoscope-guided percutaneous visible device for abdominal cavity probing and accurate drainage according to claim 2, wherein a connecting head (22) is disposed on an end surface of one end, away from the drainage tube (11), of the fluid injection tube (21) in a communicated manner, a connecting tube (23) is disposed on the connecting head (22) in a communicated manner, and an injection syringe (24) is disposed on the connecting tube (23) in a communicated manner.

4. The endoscope-guided percutaneous visible device for abdominal cavity probing and accurate drainage according to claim 3, wherein a sealing ring is disposed between the connecting head (22) and the connecting tube (23), and the sealing ring is made of rubber.

5. The endoscope-guided percutaneous visible device for abdominal cavity probing and accurate drainage according to claim 2, wherein an end surface of one end, away from the drainage tube (11), of the fluid discharge tube (31) is provided with a second fixing port (32) in a communicated manner, and a waste fluid bag is disposed on the second fixing port (32).

6. The endoscope-guided percutaneous visible device for abdominal cavity probing and accurate drainage according to claim 2, wherein a protective head (16) is disposed on the end surface of the bottom end of the drainage tube (11) in a threaded manner, and the protective head (16) is made of rubber.

7. The endoscope-guided percutaneous visible device for abdominal cavity probing and accurate drainage according to claim 6, wherein a protective cover (17) is disposed in the protective head (16), the protective cover (17) is disposed on the end surface of the bottom end of the data line (14) in a clamping manner, and the camera (15) is disposed in the protective cover (17).

8. The endoscope-guided percutaneous visible device for abdominal cavity probing and accurate drainage according to claim 1, wherein the adjusting mechanism (5) comprises a fixing base (501) fixedly mounted at the top end of the handgrip (41), a support plate (502) is fixedly mounted on a top surface of the fixing base (501), a worm (503) is rotatably disposed on the support plate (502), and a first bevel gear (504) is fixedly mounted at one end of the worm (503);
a first rotating rod (505) is rotatably disposed on the fixing base (501), a second bevel gear (506) engaged with the first bevel gear (504) is fixedly mounted at a top end of the first rotating rod (505), a first circular gear (507) is fixedly mounted at a bottom end of the first rotating rod (505), and a first gear plate (508) engaged with the first circular gear (507) is rotatably disposed on a circumferential surface of the handgrip (41);
a rotating frame (509) is further rotatably disposed on the support plate (502), a rotating shaft (510) is rotatably disposed on the rotating frame (509), and a worm wheel (511) matched with the worm (503) is fixedly mounted on a circumferential surface of the rotating shaft (510); and
a mounting rack (512) is rotatably mounted on end surfaces of two ends of the rotating shaft (510), and the mounting rack (512) is fixedly mounted at a bottom end of the display screen (42).

9. The endoscope-guided percutaneous visible device for abdominal cavity probing and accurate drainage according to claim 8, wherein a second rotating rod (513) is further rotatably disposed on the fixing base (501), a third bevel gear (514) is fixedly mounted at a top end of the second rotating rod (513), and a fourth bevel gear (515) engaged with the third bevel gear (514) is fixedly mounted on a side wall of the rotating frame (509); and
a second circular gear (516) is fixedly mounted at a bottom end of the second rotating rod (513), and a second gear plate (517) engaged with the second circular gear (516) is further rotatably disposed on a circumferential surface of the handgrip (41).

10. The endoscope-guided percutaneous visible device for abdominal cavity probing and accurate drainage according to claim 9, wherein a first knob (518) and a second knob (519) are further rotatably disposed on the circumferential surface of the handgrip (41), the first knob (518) and the second knob (519) are fixedly connected to the first gear plate (508) and the second gear plate (517) respectively, and the second knob (519) is rotatably sleeved on the first knob (518).
